(19) United States
(12) Patent Application Publication
(10) Pub. No.: US 2025/0186112 A1
(43) Pub. Date: Jun. 12, 2025

(54) MEDICAL SYSTEMS, DEVICES, AND RELATED METHODS FOR APPLYING ENERGY, DELIVERING FLUID, AND/OR MANIPULATING TISSUE

(71) Applicant: Boston Scientific Scimed, Inc., Maple Grove, MN (US)

(72) Inventor: Austin Grant JOHNSON, Worcester, MA (US)

(73) Assignee: Boston Scientific Scimed, Inc., Maple Grove, MN (US)

(21) Appl. No.: 18/967,872

(22) Filed: Dec. 4, 2024

Related U.S. Application Data
(60) Provisional application No. 63/608,399, filed on Dec. 11, 2023.

(51) Int. Cl. A61B 18/14; A61B 1/00; A61B 1/018; A61B 17/00; A61B 18/00; A61B 90/00; A61M 25/00

(52) U.S. Cl. CPC A61B 18/1492; A61B 1/00066; A61B 1/018; A61B 2017/00349; A61B 2018/00083; A61B 2018/00178; A61B 2018/00601; A61B 2018/00982; A61B 2090/034; A61B 2218/002; A61M 25/005

(57) ABSTRACT

A medical system includes a first medical device and a second medical device. The first medical device includes a first handle, a shaft, and an electrode. The first handle includes an electrical connection. The shaft extends from a distal portion of the first handle. The electrode is positioned at a distal end of the shaft. The electrode is electrically connected to the electrical connection via one or more conductive elements extending through the first handle and the shaft. The second medical device includes a second handle, a sheath, and a tissue manipulator positioned within the sheath. The sheath extends from a distal portion of the second handle. The shaft extends through at least a portion of the second handle and through the sheath.

### CROSS-REFERENCE TO RELATED APPLICATIONS

This application claims the benefit of priority to U.S. Provisional Application No. 63/608,399, filed on Dec. 11, 2023, which is incorporated by reference herein in its entirety.

### TECHNICAL FIELD

Various aspects of this disclosure generally relate to medical systems, devices, and related methods for applying energy, delivering fluid, and/or manipulating tissue. Embodiments of the disclosure relate to medical systems, devices, and related methods of treating tissue by (1) applying electrical energy to or into tissue with a distal element of a first medical device, (2) delivering or injecting fluid into, under, and/or around tissue with the distal element of the first medical device, and/or (3) manipulating tissue with a distal element of a second medical device.

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Many of these procedures involve delivering energy to tissue of an organ or a gland to treat lesions (e.g., tumors), infections, and the like. Examples of such procedures include Endoscopic Mucosal Resection (EMR), Endoscopic Sub-mucosal Resection (ESR), Endoscopic Sub-mucosal Dissection (ESD), polypectomy, mucosectomy, Peroral Endoscopic Myotomy (POEM), etc. In particular, such procedures may be carried out by inserting an insertion device into a subject's body through a surgical incision, or via a natural anatomical orifice (e.g., mouth, vagina, or rectum), and performing the procedure or operation at a treatment site with an auxiliary device inserted through the insertion device. Alternatively, the auxiliary device may be delivered to the treatment site without the insertion device.

In some procedures, a distal portion of a medical device is positioned between layers of tissue to cut, puncture, or perforate one or more layers of tissue or otherwise treat the tissue or the treatment site. In some aspects, a distal portion of the medical device is positioned between the mucosa and the muscularis layers in a portion of the gastrointestinal (GI) tract to reach a target or treatment site. Oftentimes, the positioning is achieved by (1) injecting a lifting agent into the submucosa to separate the mucosa layer from the muscularis layer and (2) utilizing a cutting knife to cut through the submucosa. In some instances, tissue that has been cut, punctured, or perforated at the treatment site may interfere with additional treatments. These concerns may increase the duration, costs, and risks of the medical procedure.

The systems, devices, and methods of this disclosure may rectify some of the deficiencies described above or address other aspects of the art.

### SUMMARY

Examples of this disclosure relate to, among other things, medical systems, devices, and methods for performing one or more medical procedures. For example, this disclosure relates to medical devices and methods for performing one of more procedures between layers of tissue. Additionally, in some examples, this disclosure relates to medical devices and methods for delivering energy (e.g., for cutting, cauterizing, perforating, puncturing, or otherwise treating tissue) and/or delivering fluid to a treatment site. Furthermore, in some examples, this disclosure relates to medical systems, devices, and methods for lifting or otherwise manipulating tissue or one or more portions of the treatment site. Each of the examples disclosed herein may include one or more of the features described in connection with any of the other disclosed examples.

In one example, a medical system may include a first medical device and a second medical device. The first medical device may include a first handle, a shaft, and an electrode. The first handle may include an electrical connection. The shaft may extend from a distal portion of the first handle. The electrode may be positioned at a distal end of the shaft. The electrode may be electrically connected to the electrical connection via one or more conductive elements extending through the first handle and the shaft. The second medical device may include a second handle, a sheath, and a tissue manipulator positioned within the sheath. The sheath may extend from a distal portion of the second handle. The shaft may extend through at least a portion of the second handle and through the sheath.

The medical system may include one or more of the following aspects. The first handle may include at least one actuation mechanism. The electrode may be movable relative to a distal end of the shaft based on movement of the at least on actuation mechanism. The second handle may include an extender and a slot. The extender may be movable within the slot to control a position of the tissue manipulator relative to a distal end of the sheath. The extender may be lockable within the slot to secure the position of the tissue manipulator relative to the distal end of the sheath. The second handle may be adjustably coupled to the first handle and/or to the shaft of the first medical device via an adjustable lock. The second handle may include one or more stop surfaces to limit a position of the second handle relative to the first handle. The second handle may be fixedly coupled to a distal end of the first handle. The tissue manipulator may be formed of and/or coated with an electrically insulative material. The sheath may be formed of a braided or coiled sheath. The sheath may include at least two adjacent lumens or channels. The at least two lumens or channels may be connected in a distal open portion of the sheath.

The tissue manipulator may include a rounded bottom surface and a flat top surface when viewed from a side and a curved shape when viewed from a distal end. The tissue manipulator may include an inwardly extending widened portion, and the tissue manipulator may include a curved shape when viewed from a distal end. The tissue manipulator may include a generally cylindrical shape. The tissue manipulator may include an outwardly extending widened at a distal end, and the tissue manipulator may include a curved shape when viewed from the distal end. The tissue manipulator may include generally cylindrical shape, and the tissue manipulator further may include a hooked portion at a distal end.

In another example, a medical system may include a first medical device and a second medical device. The first medical device may include a first handle, a shaft, and an electrode. The handle may include an electrical connection. The shaft may extend from a distal portion of the first handle. The electrode may be positioned at a distal end of the shaft. The electrode may be electrically connected to the electrical connection. The second medical device may include a second handle, a sheath, and a tissue manipulator. The sheath may extend from a distal portion of the second handle, and at least a portion of the shaft may extend through a lumen in the sheath. The tissue manipulator may be at least partially positioned within and movable relative to the sheath. The tissue manipulator may be biased toward a radially extended or bent configuration when extended distally of the sheath, such that the tissue manipulator may be positioned adjacent to and radially extended or bent away from the shaft and/or the electrode of the first medical device when the tissue manipulator is extended distally of the sheath.

The medical system may include one or more of the following aspects. The second handle may include an extender and a slot. The extender may be movable within the slot to control a position of the tissue manipulator relative to a distal end of the sheath, and the extender may be lockable within the slot to secure the position of the tissue manipulator relative to the distal end of the sheath. The sheath may include a tissue manipulator lumen to movably receive the tissue manipulator. The sheath also may include a working channel to movably receive the shaft of the first medical device, and the tissue manipulator lumen and the working channel may be connected via a distal open portion in the sheath.

In another example, a method of treating a treatment site includes delivering a distal end of a medical system to a treatment site. The medical system may include a first medical device with a shaft and an electrode movably positioned within a distal portion of the shaft. The medical system further may include a second medical device with a sheath and a tissue manipulator movably positioned within a distal portion of the sheath. The shaft is movably positioned within the sheath. The method may also include delivering energy to the treatment site by energizing the electrode to cut, puncture, or perforate one or more layers of tissue at the treatment site, and distally extending the tissue manipulator to engage with or manipulate one or more layers of tissue at the treatment site, such that the tissue manipulator extends adjacent to or distally beyond the electrode. The method may further include extending or advancing the electrode relative to the shaft and/or to the sheath, and delivering energy to the treatment site again by energizing the electrode to cut, puncture, or perforate one or more layers of tissue at the treatment site.

The method may include one or more of the following aspects. The method may also include further extending the tissue manipulator distally to engage with or manipulate one or more layers of tissue at the treatment site. The method may include further extending or advancing the electrode relative to the shaft and/or to the sheath. The method may also include delivering energy to the treatment site another time by energizing the electrode to cut, puncture, or perforate one or more layers of tissue at the treatment site.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 illustrates a perspective view of an exemplary medical device system that includes a first medical device and a second medical device, including an enlarged view of a distal portion of the medical device system in first and second configurations, according to aspects of this disclosure.

FIGS. 2A-2D illustrate the distal portion of the medical device system of FIG. 1 in various configurations relative to tissue at a treatment site, according to aspects of this disclosure.

FIGS. 3A and 3B illustrate a side view and an end or front view, respectively, of a distal portion of an exemplary second medical device, according to aspects of this disclosure.

FIGS. 4A and 4B illustrate a side view and an end or front view, respectively, of a distal portion of another exemplary second medical device, according to aspects of this disclosure.

FIGS. 5A and 5B illustrate a side view and an end or front view, respectively, of a distal portion of yet another exemplary second medical device, according to aspects of this disclosure.

FIGS. 6A and 6B illustrate a side view and an end or front view, respectively, of a distal portion of a further second medical device, according to aspects of this disclosure.

FIGS. 7A and 7B illustrate a side view and an end or front view, respectively, of a distal portion of yet a further exemplary second medical device, according to aspects of this disclosure.

FIGS. 8A and 8B illustrate a side view and an end or front view, respectively, of a distal portion of another second medical device, according to aspects of this disclosure.

FIGS. 9A and 9B illustrate an end view and a cross-sectional view, respectively, of a distal portion of the medical system, according to aspects of this disclosure.

### DETAILED DESCRIPTION

Examples of the disclosure include systems, devices, and methods for one or more of: facilitating and improving the efficacy, efficiency, and safety of treating and/or manipulating tissue when, for example, applying electrical energy to tissue with an electrode; delivering fluid into, under, and/or around tissue during a medical procedure through the distal end of the electrode; lifting, moving, or otherwise manipulating tissue; and cutting, resecting, or otherwise treating tissue. Aspects of the disclosure may provide the user with the ability to apply electrical energy or heat to tissue using a medical device having an electrode (e.g., as a distal tool), and to deliver fluid into and/or under tissue with the same medical device. Aspects of the disclosure may provide the user with the ability to apply electrical energy or heat and also deliver fluid without having to switch or swap out end effectors. Aspects of the disclosure may help the user penetrate a layer of tissue (e.g., a submucosal layer) to cause perforation or otherwise cut, cauterize, or otherwise treat tissue.

Aspects of the disclosure may help the user cut, resect, or otherwise remove tissue or other material without having to switch or swap out end effectors. Aspects of the disclosure may help the user to lift, move, or otherwise manipulate tissue (e.g., tissue that has already been cut) at or adjacent to a treatment site, which may help the user cut, resect, or otherwise remove additional tissue or other material. Aspects of the disclosure may help the user to lift, move, or otherwise manipulate tissue without having to switch or swap out end effectors. Additionally, aspects of the disclosure may help the user perform one or more of these treatments without requiring a larger insertion device and/or swapping out the medical devices, as a larger insertion device and/or swapping out medical devices may increase the procedure duration, may require multiple users, and/or may otherwise negatively affect the procedure. Moreover, aspects of the disclosure may help the user to lift, move, or otherwise manipulate tissue that has already been cut or otherwise treated, for example, such that that tissue may not interfere with or otherwise inhibit the cutting or treatment of additional tissue or other portions of the treatment site. Some aspects of the disclosure may be used in performing an endoscopic, laparoscopic, arthroscopic, gynoscopic, thoracoscopic, cystoscopic, or other type of procedure.

Embodiments of the disclosure may relate to systems, devices, and methods for performing various medical procedures and/or treating portions of the large intestine (colon), small intestine, cecum, esophagus, stomach, any other portion of the gastrointestinal tract, lungs, and/or any other suitable patient anatomy. Various embodiments described herein include single-use or disposable medical devices. Some aspects of the disclosure may be used in performing an endoscopic, arthroscopic, bronchoscopic, ureteroscopic, colonoscopic, or other type of procedure. For example, the disclosed aspects may be used with duodenoscopes, bronchoscopes, ureteroscopes, colonoscopes, gastroscopes, endoscopic ultrasonography (“EUS”) scopes, laparoscopes, arthroscopes, cystoscopes, thoracoscopes, aspiration scopes, sheaths, catheters, diagnostic or therapeutic tools or devices, or other types of medical devices. One or more of the elements discussed herein could be metallic, plastic, or include a shape memory metal (such as, e.g., Nitinol), a shape memory polymer, a polymer, or any combination of biocompatible materials.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. Proximal and distal directions are labeled with arrows marked “P” and “D”, respectively, throughout various figures. As used herein, the terms “comprises,” “comprising,” “has,” “having,” “includes,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

Reference will now be made in detail to examples of the disclosure described above and illustrated in the accompanying drawings. Wherever possible, the same reference numbers will be used throughout the drawings to refer to the same or like parts. It is noted that one or more aspects of the medical systems, devices, or methods discussed herein may be combined and/or used with one or more other aspects of the medical devices or methods discussed herein.

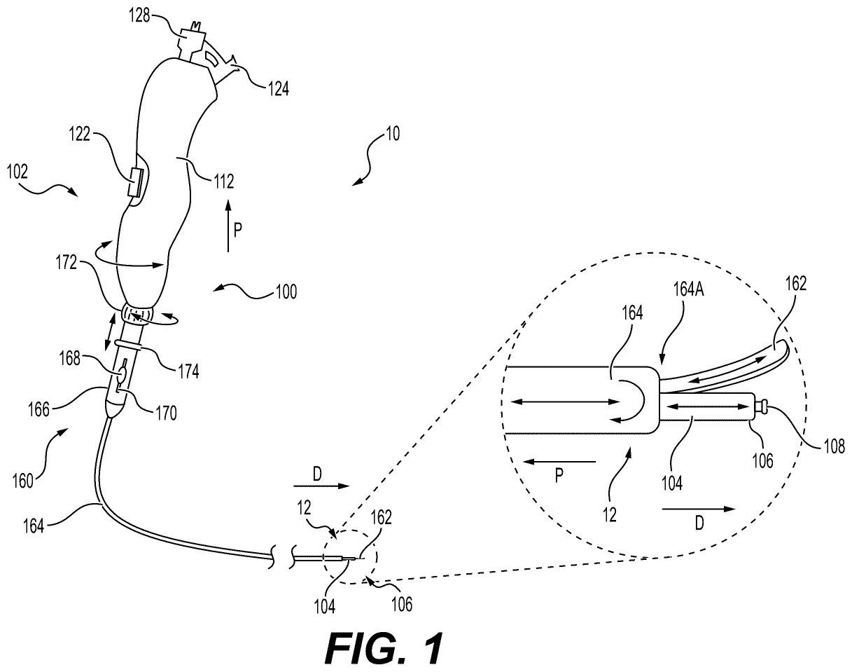

FIG. 1 illustrates a perspective view of an exemplary medical device system 10, including a first exemplary medical device 100 and a second exemplary medical device 160. First medical device 100 includes a handle 102 and a shaft 104. Shaft 104 includes a distal end 106. First medical device 100 also includes a tool, for example, an electrode 108 at distal end 106. Electrode 108 may be movable (e.g., longitudinally extendable and retractable) relative to shaft 104, for example, extendable and/or retractable longitudinally relative to distal end 106 of shaft 104. Electrode 108 may be energized to puncture, perforate, cut, resect, cauterize, remove, or otherwise treat tissue or other portions of a treatment site. In some aspects, although not shown, electrode 108 may include an electrode lumen or a central lumen that terminates in an electrode outlet or electrode opening to inject or otherwise deliver fluid to the treatment site, for example, into or between layers of tissue. In other aspects, however, electrode 108 may not include an electrode lumen, and thus may not provide for injection or other delivery of fluid.

Second medical device 160 includes a distal tool, an end effector, or a tissue manipulator 162, for example, in the form of a bar or rod. Second medical device 160 also includes a sheath 164 and a handle 166. At least a portion of tissue manipulator 162 may be movably positioned within sheath 164, for example, extendable distally of a distal end 162A of sheath 164. A proximal end of sheath 164 may be coupled to a distal end of handle 166. Additionally, handle 166 may include an actuation mechanism or extender 168 and a slot 170. As discussed in detail below, tissue manipulator 162 may be coupled to extender 168, and extender 168 may be movable within slot 170 to control the position and/or configuration of tissue manipulator 162, for example, relative to distal end 164A of sheath 164. Furthermore, second medical device 160 may be coupled to first medical device 100. For example, shaft 104 of first medical device 100 may be positioned radially within sheath 164, and one or more portions of second medical device 160 (e.g., sheath 164 and/or handle 166) may be selectively secured or coupled to handle 102 and/or shaft 104. In some aspects, first medical device 100 and second medical device 160 may be separate devices, for example, moveable relative to one another. In other aspects, first medical device 100 and second medical device 160 may be integrally formed, for example, affixed or otherwise formed together.

As shown in FIG. 1, handle 102 includes a handle body 112. A distal portion of handle body 112 may be coupled to a proximal portion of shaft 104 via a connector or strain relief element (not shown). Additionally, handle 102 includes one or more actuators or control mechanisms. For example, handle 102 may include an actuation mechanism, button, actuator, or trigger 122, for example, movable relative to a distal portion of handle body 112. Trigger 122 may be movable, for example, slideable (e.g., longitudinally movable), actuatable, or depressible, to control a position (e.g., extension and/or retraction) and/or energization of electrode 108. For example, sliding, actuating, or depressing trigger 122 may extend electrode 108 from distal end 106 of shaft 104. In these aspects, electrode 108 may be extendable from distal end 106 of shaft 104 by approximately 5.0 mm to approximately 2.0 cm. Trigger 122 may be biased (e.g., spring-biased), such that trigger 122 may return to the proximal or non-depressed position once pressure on trigger 122 is no longer applied. Returning to the proximal or non-depressed position may also retract electrode 108 to the non-extended position. Alternatively or additionally, in some aspects, sliding, actuating, or depressing trigger 122 may control the energization of electrode 108. For example, depressing trigger 122 may energize electrode 108, and releasing pressure on trigger 122 may terminate the energizing of electrode 108. In other aspects, the energization of electrode 108 may be controlled by a different button, actuator, or trigger, for example, via a foot pedal.

Handle 102 also includes one or more connections and/or ports, for example, extending from or otherwise coupled to handle body 112. For example, handle 102 may include one or more of an electrode fluid port 124 and/or an electrical connection 128. As discussed below, electrode fluid port 124 may be fluidly connected to electrode 108, for example, via one or more lumens in handle 102 and shaft 104. Electrical connection 128 may be electrically connected to electrode 108, for example, via one or more conductive elements (e.g., wires, cables, filaments, rods, etc.) in handle 102 and shaft 104.

Electrode fluid port 124 and/or electrical connection 128 may be positioned on respective proximal portions of handle body 112, for example, adjacent or proximal to knobs 116 and 118. Electrode fluid port 124 may receive fluid (e.g., water, Orise™ gel, saline, etc.) to be delivered via a lumen in electrode 108, for example, between two layers of tissue or otherwise to the treatment site. The delivery of the fluid to each of electrode fluid port 124 may be controlled by a fluid delivery device, for example, a syringe, pump, etc. Moreover, electrical connection 128 may receive energy (e.g., cautery energy) from an energy source to be delivered to electrode 108, for example, to cut, cauterize, penetrate, perforate, or otherwise treat tissue. In some aspects, the delivery of energy to electrical connection 128 may be controllable, for example, via a separate button or trigger, one or more foot pedals, etc.

Furthermore, although not shown, handle 102 may include a strap or other connection element, for example, coupled to or otherwise extending from handle body 112. In some aspects, the strap or other connection element may help to allow for handle body 112 to be coupled to another medical device, for example, to a handle or other portion of a scope or other insertion device.

As mentioned, second medical device 160 may include a tissue manipulator 162, for example, at a distal end of second medical device 160. Second medical device 160 also includes sheath 164 (e.g., an outer sheath). Shaft 104 may be at least partially positioned within a lumen of sheath 164. A proximal portion of sheath 164 may include or otherwise be coupled to handle 166 or another control mechanism. Handle 166 may include a knob or extender 168, for example, that is movable (e.g., longitudinally movable) within slot 170 in handle 166. The position of extender 168 may control the position of tissue manipulator 162, for example, relative to a distal end 164A of sheath 164. In some aspects, extender 168 may be lockable or otherwise securable within slot 170, for example, by rotating extender 168, by a friction fit, etc. The locked or otherwise secured position of extender 168 within slot 170 may also help to lock or otherwise secure the position of tissue manipulator 162 relative to distal end 164A of sheath 164. Additionally, although not shown, in some aspects, second medical device 160 may include one or more springs or biasing elements, for example, in handle 166, to bias extender 168 (and thus tissue manipulator 162) toward a proximal or at least partially retracted position or configuration.

In some aspects, second medical device 160 may be secured to first medical device 100 via one or more locks 172. The one or more locks 172 may be Touhy-Borst locks. The one or more locks 172 may help to secure a proximal portion of sheath 164 and/or a proximal portion of handle 166 to shaft 104 and/or handle 102 (e.g., to the strain relief portion) of first medical device 100. For example, lock(s) 172 may be rotatable in one direction (e.g., clockwise) to help secure second medical device 160 to first medical device 100. Additionally, lock(s) 172 may be rotatable in another direction (e.g., counter-clockwise) to help unsecure/detach second medical device 160 from first medical device 100, for example, to adjust the relative position(s) of second medical device 160 and first medical device 100.

Furthermore, in some aspects, one or more portions of second medical device 160 may include one or more stoppers or stop surfaces 174. For example, one or more portions of handle 166 may include an enlarged surface (e.g., larger diameter) that may for stop surface 174 help to limit the position or movement of second medical device 160 relative to first medical device 100. In other aspects, one or more portions of handle 166 may include one or more knobs, for example, to lock, limit, or otherwise control the movement and/or relative positioning between first medical device 100 and second medical device 160. In these examples, stop surface(s) 174 may help to prevent lock(s) 172 from being positioned distal to stop surface(s) 174. Stated another way, stop surface(s) 174 may help to prevent the portion of second medical device 160 that includes stop surface(s) 174 (e.g., a portion of handle 166) from being positioned proximal to lock(s) 172. Nevertheless, in any of these aspects, shaft 104 of first medical device 100 may be movable (e.g., longitudinally movable) relative to sheath 164, and/or sheath 164 may be movable (e.g., longitudinally movable) relative to shaft 104. For example, distal end 106 of shaft 104 (e.g., including electrode 108) may be extendable from distal end 164A of sheath 164 through a range of approximately 0 cm to approximately 20.0 cm, for example, up to approximately 5.0 cm, up to approximately 3.0 cm, etc.

In other aspects, one or more portions of second medical device 160 may be integrally formed or otherwise coupled, for example, affixed or otherwise formed together with one or more portions of first medical device 100. For example, handle 166 of second medical device 160 may be integrated with or otherwise coupled to (e.g., welded, adhered, integrally formed from a same piece of material, etc.) handle 102 of first medical device 100. In one aspect, a proximal portion of handle 166 may be fixedly coupled to a distal portion of handle 102. If handle 166 is fixedly coupled to handle 102, then handle 166 may not include one or more stoppers or stop surfaces 174.

Tissue manipulator 162 may be extendable, for example, to help lift tissue (e.g., cut tissue) or other portions of a treatment site. Although not shown, tissue manipulator 162 may be coupled to knob or extender 168, for example, via one or more couplers (e.g., rod(s), shaft(s), wire(s), drive wire(s), etc.). A distal end of the one or more couplers may be coupled (e.g., welded, adhered, press fit, crimped, etc.) to a proximal end of tissue manipulator 162. In these aspects, the position of extender 168 within or along slot 170 may help to control the position of tissue manipulator 162, for example, the position of a distal portion of tissue manipulator 162 relative to distal end 164A of shaft 164. For example, extender 168 may control the position of tissue manipulator 162 relative to distal end 164A of shaft 164. In these aspects, tissue manipulator 162 may be movable (e.g., longitudinally movable) between, at least, a position in which tissue manipulator 162 is at least partially retracted (e.g., proximally of distal end 164A of shaft 164) and one or more positions in which tissue manipulator 162 is extended (e.g., distally of distal end 164A of shaft 164). For example, tissue manipulator 162 may be extended by approximately 0 cm to approximately 20.0 cm from distal end 164A, for example, by approximately 3.0 cm to approximately 5.0 cm. In some aspects, tissue manipulator 162 may be extended distally of distal end 164A by approximately 5.0 mm to approximately 2.0 cm.

Additionally, at least a distal portion of tissue manipulator 162 may be biased radially outward (e.g., away from electrode 108 of first medical device) to help urge tissue away from first medical device 100 (e.g., away from electrode 108). In some aspects, tissue manipulator 162 may be formed of one or more materials, for example, Nitinol, a plastic material, a steel (e.g., stainless steel), etc. For example, at least a distal portion of tissue manipulator 162 may be able to deform, such that tissue manipulator 162 may be received within sheath 164, and at least the distal portion of tissue manipulator 162 may expand and/or move radially outward when extended distally of sheath 164. In these aspects, at least a portion of tissue manipulator 162 may be formed of a steel, and may be stamped into the desired pre-set shape. In some other aspects, at least a portion of tissue manipulator 162 may be formed of a shape memory material (e.g., Nitinol, plastic, etc.), and may be shape set into the desired pre-set shape.

Furthermore, one or more portions of tissue manipulator 162, one or more portions of the one or more couplers, and/or one or more portions that couple tissue manipulator 162 and the one or more couplers may be insulative or non-conductive (e.g., formed of a plastic, elastomer, etc.). In some aspects, one or more portions of or an entirety of tissue manipulator 162 may be formed of a plastic or other material that is not electrically conductive. In another example, one or more portions of or an entirety of tissue manipulator 162 may be coated in an insulative or otherwise non-conductive material (e.g., a dielectric coating). In some examples, tissue manipulator 162 may be formed of a metallic material (e.g., stainless steel, Nitinol, etc.), for example, to impart a desired shape, stiffness (e.g., for when pushing on or otherwise interacting with tissue), shape memory, etc., and tissue manipulator 162 may be coated with coating or material that is not electrically conductive (e.g., the dielectric coating). In other examples, the coupler(s) may be formed of a plastic or other material that is not electrically conductive. In these aspects, energy delivered to a treatment site (e.g., via electrode 108) may not be conveyed proximally and/or otherwise applied to the treatment site (e.g., other portions or locations of the treatment site or adjacent to the treatment site) via tissue manipulator 162 and/or the one or more couplers.

In some aspects, second medical device 160 may be rotatable, for example, along with the rotation of first medical device 102 or separately rotatable from first medical device 102. In examples, second medical device 160 may be rotatable relative to first medical device 102. For example, second medical device 160 may be secured to or otherwise coupled to first medical device 100, and first medical device 100 and second medical device 160 may be rotated together, thus rotating shaft 104 (including distal end 106), electrode 108, sheath 164, tissue manipulator 162, etc. In some aspects, second medical device 160 may be unsecured from first medical device 100 (e.g., via loosening or uncoupling lock(s) 172), and second medical device 160 may be rotated, for example, via rotation of handle 166. In any of these aspects, the structure, material, or other aspects of sheath 164 may help to convey rotation, for example, from handle 166 to distal end 164A and tissue manipulator 162. For example, in some aspects, sheath 164 may be formed of or include a braided sheath, one or more coils, one or more sheathed braids or coils, an extruded sheath, an extruded coil, etc.

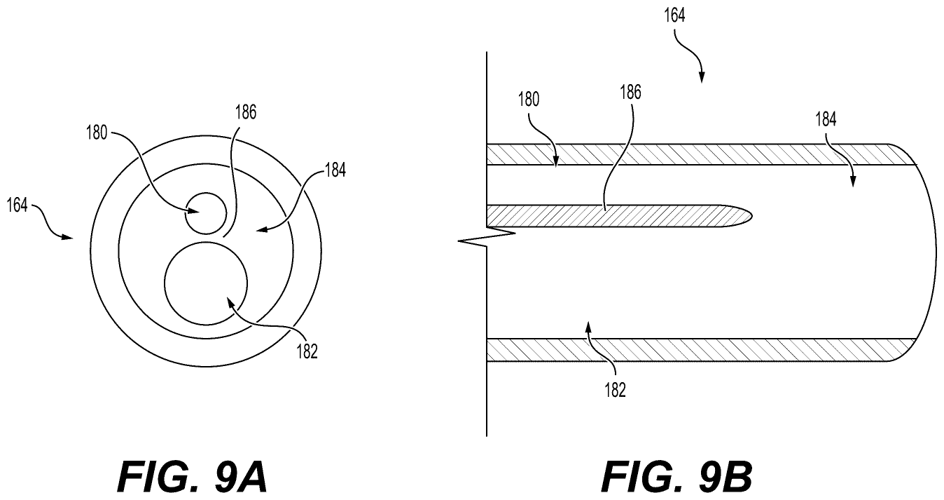

Additionally, in some aspects, as shown in FIGS. 9A and 9B, sheath 164 may include a plurality of lumens or grooves. In some aspects, sheath 164 may include a tissue manipulator lumen 180, for example, to movably receive tissue manipulator 162 (FIG. 1) and/or the one or more couplers that couple tissue manipulator 162 to extender 168 (FIG. 1). Sheath 164 may also include a main lumen 182 or working channel, for example, to movably receive shaft 104 (FIG. 1) of first medical device 100. Tissue manipulator lumen 180 and main lumen 182 may be at least partially separated by a wall 186. In these aspects, tissue manipulator lumen 180 and/or main lumen 182 may help to ensure that tissue manipulator 162 rotates as sheath 164 rotates.

Alternatively or additionally, one or more portions of tissue manipulator 162 and/or the coupler(s) coupling tissue manipulator 162 to extender 168 may help to ensure that tissue manipulator 162 rotates with the rotation of proximal portions of second medical device 160 (e.g., with the rotation of handle 166). For example, one or more portions of tissue manipulator 162 and/or the coupler(s) may include one or more flat or contoured surfaces. For example, the coupler(s) may include a flat surface, may be generally triangular, square, etc. Alternatively or additionally, one or more portions of tissue manipulator 162 and/or the coupler(s) may include a frictional surface (e.g., formed by a frictional coating) to help ensure that tissue manipulator and/or the coupler(s) rotate with the rotation of sheath 164 (e.g., as controlled by rotation of handle 166). In any of these aspects, the rotation of tissue manipulator 162 may help for the user to position tissue manipulator 162 relative to the treatment site, for example, to help lift cut tissue or other portions of the treatment site.

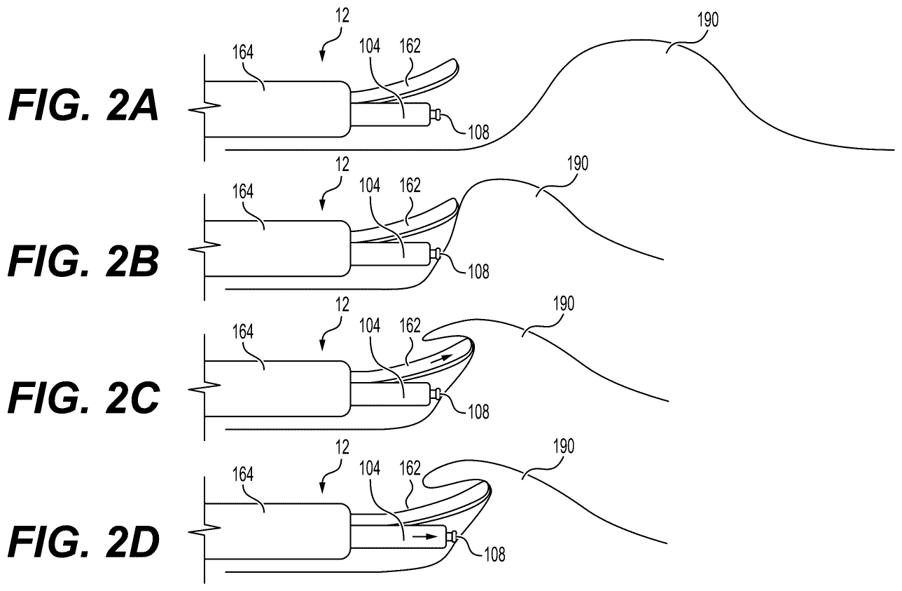

FIGS. 2A-2D illustrate a distal portion 12 of medical device system 10 in various configurations or method steps relative to tissue at a treatment site to help apply energy, deliver fluid, and/or manipulate tissue. As shown in FIG. 2A, in an initial step, distal portion 12 may be positioned at or adjacent to a treatment site, for example, an ESD injection bleb 190. In these aspects, shaft 104 and electrode 108 may be extended distally of sheath 164. In some aspects, although not shown, electrode 108 may include a fluid lumen, and fluid may be delivered into tissue via the fluid lumen of electrode 108 to help form bleb 190. Furthermore, tissue manipulator 162 may be extended at least partially distally of sheath 164, for example, adjacent to distal end 106 of shaft 104.

As shown in FIG. 2B, in a next step, one or more aspects of distal portion 12 may be moved (e.g., advanced distally) to make an initial cut, penetration, perforation or incision at the treatment site (e.g., bleb 190). For example, first medical device 100, including shaft 104 and electrode 108, may be advanced distally. Alternatively or additionally, first medical device 100 and second medical device 160 (e.g., tissue manipulator 162 and sheath 164) may both be advanced distally. In these aspects, electrode 108 may be extended distally of distal end 106 of shaft 104 and/or may be energized. When energized, electrode 108 may be positioned adjacent bleb 190 to apply or deliver energy to tissue, for example, to at least partially cut the tissue of bleb 190. Furthermore, tissue manipulator 162 may be positioned adjacent to electrode 108, for example, helping to lift tissue (e.g., cut tissue) adjacent to electrode 108.

FIG. 2C illustrates a next step, which includes moving (e.g., distally extending) tissue manipulator 162. In these aspects, tissue manipulator 162 may be extended distally of sheath 164 (e.g., by advancing extender 168 distally within slot 170 of handle 166, FIG. 1). Additionally, as shown, tissue manipulator 162 may extend radially outward as tissue manipulator 162 is extended distally of sheath 164. At least a portion of tissue manipulator 162 (e.g., a distal end or distal portion) may abut, engage, or otherwise interact with tissue (e.g., cut tissue of bleb 190), and tissue manipulator 162 may help to lift or otherwise urge the tissue distally and/or radially away from electrode 108, shaft 104, and/or other portions of first medical device 100.

FIG. 2D illustrates another step, which includes further cutting the tissue of bleb 190 and/or advancing electrode 108. In some aspects, the step shown in FIG. 2D may include advancing one or more components of medical system 10, for example, advancing one or more of handle 102 and handle 166 distally to move shaft 104 (including distal end 106), electrode 108, tissue manipulator 162, and/or sheath 164 distally. In some aspects, the step shown in FIG. 2D may include moving first medical device 100 relative to second medical device 160, and thus advancing shaft 104 (including distal end 106) and electrode 108 distally relative to tissue manipulator 162 and/or sheath 164. For example, lock(s) 172 (FIG. 1) may be unsecured, such that second medical device 160 may be moved relative to first medical device 100. In these aspects, electrode 108 may be extended distally of distal end 106 of shaft 104 and/or may be energized. When energized, electrode 108 may again be positioned adjacent bleb 190 to apply or deliver energy to tissue, for example, to again at least partially cut the tissue of bleb 190. Additionally or alternatively, in some aspects, the position of tissue manipulator 162 may be secured, for example, relative to sheath 164. As mentioned above, extender 168 may be secured within slot 170 of handle 166, for example, to help secure the position of tissue manipulator 162 relative to sheath 164. In these aspects, tissue manipulator 162 may help to lift or otherwise urge the tissue distally and/or radially away from electrode 108, shaft 104, and/or other portions of first medical device 100 as electrode 108 cuts or otherwise applies energy to bleb 190 or other portions of the treatment site.

It is noted that one or more steps of FIGS. 2A-2D may be repeated as many times as necessary, for example, to help remove the tissue of bleb 190 or otherwise treat the treatment site. For example, one or more of shaft 104, electrode 108, tissue manipulator 162, and/or sheath 164 may be advanced distally or otherwise manipulated relative to the treatment site to incrementally cut the tissue of bleb 190 until bleb 190 is removed from the treatment site.

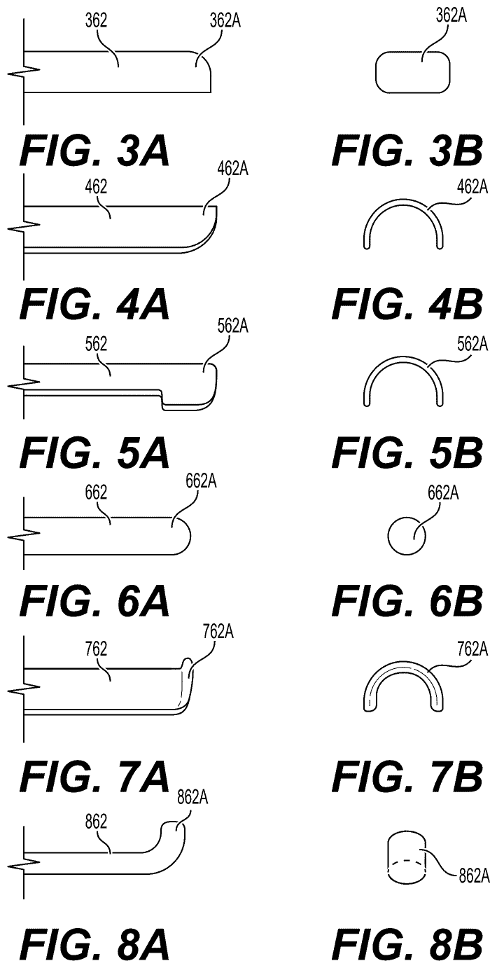

FIGS. 3A-8B illustrate distal portions of additional exemplary tissue manipulators. It is noted that any of the tissue manipulators illustrates in FIGS. 3A-8B may be as or in the place of tissue manipulator 162.

FIGS. 3A and 3B illustrate a side view and an end or front view, respectively, of a distal portion of an exemplary second medical device, including a tissue manipulator 362. As shown in FIG. 3A, tissue manipulator 362 may be generally rectangular in its side view. For example, a distal end 362A of tissue manipulator 362 may include a flat outer portion and a rounded inner portion. As shown in FIG. 3B, distal end 362A may include a generally rectangular shape, for example, including a generally flat top, a generally flat bottom, and generally flat sides.

FIGS. 4A and 4B illustrate a side view and an end or front view, respectively, of a distal portion of an exemplary second medical device, including a tissue manipulator 462. As shown in FIG. 4A, tissue manipulator 462 may be generally rectangular in its side view. For example, a distal end 462A of tissue manipulator 462 may include a flat outer portion and a rounded inner portion. As shown in FIG. 4B, distal end 462A may include a generally rounded or semi-circular shape, for example, with a curved bottom surface and/or a curved top surface.

FIGS. 5A and 5B illustrate a side view and an end or front view, respectively, of a distal portion of an exemplary second medical device, including a tissue manipulator 562. As shown in FIG. 5A, a proximal portion of tissue manipulator 562 may be generally rectangular in its side view, and a distal portion or distal end 562A of tissue manipulator 562 may include a widened portion. For example, distal end 562A may extend downward (or radially inward) toward first medical device 100 (e.g., toward electrode 108). Additionally, various portions of tissue manipulator 562, for example, including distal end 562A, may include a flat outer portion and one or more rounded inner portions. As shown in FIG. 5B, distal end 562A may include a generally rounded or semi-circular shape, for example, with a curved bottom surface and/or a curved top surface.

FIGS. 6A and 6B illustrate a side view and an end or front view, respectively, of a distal portion of an exemplary second medical device, including a tissue manipulator 662. As shown in FIG. 6A, tissue manipulator 662 may be generally cylindrical or rod-shaped in its side view. For example, as shown in FIG. 6B, a distal end 662A of tissue manipulator 662 may include a generally circular shape.

Alternatively, although not shown, distal end 662A may alternatively include a generally oval, elliptical, or otherwise rounded shape.

FIGS. 7A and 7B illustrate a side view and an end or front view, respectively, of a distal portion of an exemplary second medical device, including a tissue manipulator 762. As shown in FIG. 7A, a proximal portion of tissue manipulator 762 may be generally rectangular in its side view, and a distal portion or distal end 762A of tissue manipulator 762 may include a widened portion. For example, distal end 762A may extend upward (or radially upward) away from first medical device 100 (e.g., away from electrode 108) to include a flared out portion. Additionally, various portions of tissue manipulator 762, for example, including distal end 762A, may include a flat outer portion and one or more rounded inner portions. As shown in FIG. 7B, distal end 762A may include a generally rounded or semi-circular shape, for example, with a curved bottom (or radially inner) surface and/or a curved top (or radially outer) surface. It is noted that the thickness of distal end 762A may be thicker than a thickness of the distal ends of other tissue manipulators (see, e.g., distal ends 462A and 562A), for example, due to the flared out portion. In some aspects, the flared out portion may be retractable within distal end 164A of sheath 164. In other aspects, the flared out portion may not be retractable within distal end 164A of sheath 164, for example, forming a stop surface that helps to limit the retraction of tissue manipulator 762 relative to sheath 164.

FIGS. 8A and 8B illustrate a side view and an end or front view, respectively, of a distal portion of an exemplary second medical device, including a tissue manipulator 862. As shown in FIG. 8A, a proximal portion of tissue manipulator 862 may be generally cylindrical in its side view, and a distal portion or distal end 862A of tissue manipulator 862 may include a widened or curved portion. For example, distal end 862A may extend upward (or radially upward) away from first medical device 100 (e.g., away from electrode 108) to include a hook or hooked portion. Additionally, various portions of tissue manipulator 862, for example, including distal end 862A, may include a flat outer portion and one or more rounded inner portions. As shown in FIG. 8B, distal end 862A may include a bottom (or radially inner) surface may include generally curved, rounded, or semi-circular shape, and distal end 862A may include a flat top (or radially outer) surface. Alternatively, although not shown, distal end 862A may include a top (or radially outer) surface with a generally, curved, rounded, or semi-circular shape. It is noted that the thickness of distal end 862A may be thicker than a thickness of the distal ends of other tissue manipulators (see, e.g., distal ends 662A), for example, due to the hook or hooked portion. In some aspects, hook or hooked portion may be retractable within distal end 164A of sheath 164. In other aspects, the hook or hooked portion may not be retractable within distal end 164A of sheath 164, for example, forming a stop surface that helps to limit the retraction of tissue manipulator 862 relative to sheath 164.

It is noted that tissue manipulators 362, 462, 562, 662, 762, and 862 are illustrated in a retained or constrained configuration, that is, in a straight configuration for when the tissue manipulators are at least partially constrained or retained within sheath 164. It is further noted that tissue manipulators 362, 462, 562, 662, 762, and 862 may bend or otherwise change shape such that at least respective distal portions extend radially outward when exposed from sheath 164 (e.g., be biased toward a radially extended or bent configuration). The radius of curvature of tissue manipulators 162, 362, 462, 562, 662, 762, and 862 may vary, for example, between approximately 0 degrees to approximately 90 degrees. Additionally, the radius of curvature of tissue manipulators 162, 362, 462, 562, 662, 762, and 862 may depend on the amount (e.g., portion of a length) of the tissue manipulator that is exposed distally beyond distal end 164A of sheath 164.

Although not shown, the tissue manipulator may include one or more other shapes or sizes, which may help the tissue manipulator push, engage with, or otherwise manipulate tissue at a treatment site. The various shapes and/or configurations of the various tissue manipulators may help to promote a grip on tissue when engaged with tissue (e.g., cut tissue of bleb 190). For example, the distal ends (e.g., the distal end faces) of the tissue manipulators may include one or more points, gripping features (e.g., surface texture, spikes, bumps, etc.), or other features to catch or otherwise interact with and/or manipulate tissue. Additionally or alternatively, the various shapes and/or configurations of the various tissue manipulators may help to promote a shared or abutting surface area when engaged with the tissue, for example, to help push or otherwise urge the tissue away from electrode 108 or other aspects of first medical device 100.

As mentioned above, FIGS. 9A and 9B, sheath 164 may include a plurality of lumens, channels, or grooves. In some aspects, sheath 164 may include tissue manipulator lumen 180, for example, to movably receive tissue manipulator 162 and/or the one or more couplers that couple tissue manipulator 162 to extender 168. Sheath 164 may also include main lumen 182 or working channel, for example, to movably receive shaft 104 of first medical device 100. Tissue manipulator lumen 180 and main lumen 182 may be at least partially separated by wall 186. Additionally, tissue manipulator lumen 180 and main lumen 182 may be connected or otherwise in communication via a distal open portion 184. Open portion 184 may include a length that is slightly longer than a length of the tissue manipulator (e.g., tissue manipulator 162), such that the tissue manipulator may be fully retracted with sheath 164 and positioned within open portion 184.

Various aspects discussed herein may allow for one or more medical devices (e.g., first medical device 100 and second medical device 160) to be delivered to a treatment site, for example, alone or via a scope or insertion device, to perform endoscopic submucosal dissection (“ESD”) or otherwise treat a treatment site. Various aspects discussed herein may allow for a user to engage with or otherwise manipulate tissue (e.g., cut tissue) at the treatment site, which may help the user to further cut or otherwise treat the treatment site with a reduced risk of otherwise inadvertently contacting unintended tissue or unintended portions of the treatment site. Various aspects discussed herein may help to allow for the user to more effectively or efficiently apply energy, cut, or otherwise treat tissue, for example, with a reduced likelihood of cut tissue or other aspects of the treatment site interfering with the treatment. Additionally, various aspects discussed herein may help to improve the efficacy of treatment and/or recovery from a procedure, for example, a procedure to treat a treatment side. Various aspects discussed herein may help to reduce and/or minimize the duration of the procedure, and/or may help reduce risks of inadvertent contact with tissue or other material during delivery, repositioning, or removal of a medical device for the procedure.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical system, comprising:
a first medical device, including:
a first handle, including an electrical connection;
a shaft extending from a distal portion of the first handle; and
an electrode positioned at a distal end of the shaft, wherein the electrode is electrically connected to the electrical connection via one or more conductive elements extending through the first handle and the shaft; and
a second medical device, including:
a second handle;
a sheath extending from a distal portion of the second handle; and
a tissue manipulator positioned within the sheath,
wherein the shaft extends through at least a portion of the second handle and through the sheath.

2. The medical system of claim 1, wherein the first handle includes at least one actuation mechanism, and wherein the electrode is movable relative to a distal end of the shaft based on movement of the at least on actuation mechanism.

3. The medical system of claim 1, wherein the second handle includes an extender and a slot, and wherein the extender is movable within the slot to control a position of the tissue manipulator relative to a distal end of the sheath.

4. The medical system of claim 3, wherein the extender is lockable within the slot to secure the position of the tissue manipulator relative to the distal end of the sheath.

5. The medical system of claim 1, wherein the second handle is adjustably coupled to the first handle and/or to the shaft of the first medical device via an adjustable lock.

6. The medical system of claim 5, wherein the second handle includes one or more stop surfaces to limit a position of the second handle relative to the first handle.

7. The medical system of claim 1, wherein the second handle is fixedly coupled to a distal end of the first handle.

8. The medical system of claim 1, wherein the tissue manipulator is formed of and/or coated with an electrically insulative material.

9. The medical system of claim 1, wherein the sheath is formed of a braided or coiled sheath.

10. The medical system of claim 1, wherein the sheath includes at least two adjacent lumens or channels, wherein the at least two lumens or channels are connected in a distal open portion of the sheath.

11. The medical system of claim 1, wherein the tissue manipulator includes a rounded bottom surface and a flat top surface when viewed from a side and a curved shape when viewed from a distal end.

12. The medical system of claim 1, wherein the tissue manipulator includes an inwardly extending widened portion, and wherein the tissue manipulator includes a curved shape when viewed from a distal end.

13. The medical system of claim 1, wherein the tissue manipulator includes a generally cylindrical shape.

14. The medical system of claim 1, wherein the tissue manipulator includes an outwardly extending widened at a distal end, and wherein the tissue manipulator includes a curved shape when viewed from the distal end.

15. The medical system of claim 1, wherein the tissue manipulator includes generally cylindrical shape, and wherein the tissue manipulator further includes a hooked portion at a distal end.

16. A medical system, comprising:
a first medical device, including:
a first handle, including an electrical connection;
a shaft extending from a distal portion of the first handle; and
an electrode positioned at a distal end of the shaft, wherein the electrode is electrically connected to the electrical connection; and
a second medical device, including:
a second handle;
a sheath extending from a distal portion of the second handle, wherein at least a portion of the shaft extends through a lumen in the sheath; and
a tissue manipulator at least partially positioned within and movable relative to the sheath, wherein the tissue manipulator is biased toward a radially extended or bent configuration when extended distally of the sheath, such that the tissue manipulator is positioned adjacent to and radially extended or bent away from the shaft and/or the electrode of the first medical device when the tissue manipulator is extended distally of the sheath.

17. The medical system of claim 16, wherein the second handle includes an extender and a slot, wherein the extender is movable within the slot to control a position of the tissue manipulator relative to a distal end of the sheath, and wherein the extender is lockable within the slot to secure the position of the tissue manipulator relative to the distal end of the sheath.

18. The medical system of claim 16, wherein the sheath includes a tissue manipulator lumen to movably receive the tissue manipulator, wherein the sheath also includes a working channel to movably receive the shaft of the first medical device, and wherein the tissue manipulator lumen and the working channel are connected via a distal open portion in the sheath.

19. A method of treating a treatment site, comprising:
delivering a distal end of a medical system to a treatment site, wherein the medical system includes a first medical device with a shaft and an electrode movably positioned within a distal portion of the shaft, wherein the medical system further includes a second medical device with a sheath and a tissue manipulator movably positioned within a distal portion of the sheath, and wherein the shaft is movably positioned within the sheath;
delivering energy to the treatment site by energizing the electrode to cut, puncture, or perforate one or more layers of tissue at the treatment site;
distally extending the tissue manipulator to engage with or manipulate one or more layers of tissue at the treatment site, such that the tissue manipulator extends adjacent to or distally beyond the electrode;
extending or advancing the electrode relative to the shaft and/or to the sheath; and
delivering energy to the treatment site again by energizing the electrode to cut, puncture, or perforate one or more layers of tissue at the treatment site.

20. The method of claim 19, further comprising:
further extending the tissue manipulator distally to engage with or manipulate one or more layers of tissue at the treatment site;
further extending or advancing the electrode relative to the shaft and/or to the sheath; and
delivering energy to the treatment site another time by energizing the electrode to cut, puncture, or perforate one or more layers of tissue at the treatment site.
